(19) United States
(12) Patent Application Publication
(10) Pub. No.: US 2007/0049860 A1
(43) Pub. Date: Mar. 1, 2007

(54) Apparatus and method for using a surgical instrument with an expandable sponge

(76) Inventor: Robert Seminara, Brooklyn, NY (US)

Correspondence Address: MORGAN & FINNEGAN, L.L.P., 3 World Financial Center, New York, NY, 10281-2101, US

(21) Appl. No.: 11/219,423

(22) Filed: Sep. 1, 2005

(51) Int. Cl. A61M 35/00

(52) U.S. Cl. 604/1

(57) ABSTRACT

An apparatus and method for using a surgical instrument to quickly, safely and effectively clear tissue, procure dissection and contain bleeding during surgery. The instrument comprises an expandable sponge suitable for insertion into a body cavity. The sponge comprises a material having a first unexpanded state and a second expanded state. The expandable sponge may be attached to the distal end of a handle. The expandable sponge may have slots or filet cuts which, when the sponge expands, become a network of ridges and grooves to increase the surface area of the sponge and entrap tissue for dissection. The expandable sponge may also have an indented neck region adapted to engage blood vessels and ductal structures encountered in most surgical procedures. The sponge may have taper angles at the proximal end of the sponge and at the distal end region to facilitate insertion into and removal from a trocar, introducer or cannula.

### FIELD OF THE INVENTION

This invention relates generally to surgical instruments and procedures and more particularly to physiologically compatible expandable sponges for use in surgery. The invention relates to an expandable sponge with a first unexpanded state and a second expanded state. The sponge is specially configured to efficiently clear tissue from surgically important structures and to effectively contain bleeding, thus maintaining a clear, safe field during surgery. The expandable sponge may be attached to a handle and is designed to be easily manipulated by the surgeon. The expandable sponge is designed to be used in conjunction with a trocar or cannula.

### BACKGROUND OF THE INVENTION

As laparoscopic instruments and techniques have become safer, laparoscopic surgery has become accepted as a viable alternative for the treatment of a wide array of conditions. Laparoscopy often provides a safer alternative then older procedures. Where older surgical techniques required relatively large incisions, laparoscopic procedures require relatively small surgical incisions for the surgeon to remotely manipulate surgical instruments. In endoscopic and laparoscopic surgical procedures, the surgeon makes a small incision in the patient to provide access for a trocar or cannula device. The cannula allows the surgeon to insert various surgical instruments such as scissors, forceps or dissectors. Therefore, instruments for laparoscopic surgery are designed to fit through a trocar, introducer or cannula.

In most surgical operations, the surgeon must clear tissue from organs and blood vessels and limit bleeding to provide visualization of, and access to, anatomical structures. Surgically important structures such as arteries, ducts and other important vessels, as well as various organs are often found within adipose tissue and mesocholonic or mesothelial tissue. Laparoscopic surgeons generally rely on vision to identify these structures, so it is critical to establish and maintain a clear visual field in which to work. As in most surgery, time is of the essence in laparascopic procedures, and tissue must be cleared quickly and efficiently.

It has been common in laparoscopic procedures for surgeons to use scissors to clear tissue. However, scissors can damage adjacent anatomical structures and cause trauma to surrounding tissue. Recently, laparoscopic surgeons have used blunt dissecting instruments, the most common of which is the “peanut” gauze dissector. Such dissectors generally comprise an elongate 5 or 10 mm diameter rod with a blunt tip at the end. The tip is either spherical or cylindrical and is formed of wound cotton or other fibrous material.

The “peanut” dissectors were designed to reduce the risk of traumatic injury to adjacent anatomical structures. However, because the “peanut” dissectors have only a small narrow tip of fibrous material, these dissectors are not efficient for the abrasive removal of tissue. The small tip does not provide sufficient surface area to engage the tissue, nor does it provide the structural configuration or an appropriate design to promote adherence of tissue to the tip. Furthermore, the “peanut” dissector's tip is small and therefore inadequate for containing bleeding. The small tip of the “peanut” dissector provides insufficient padding to protect anatomical structures from traumatic injury. A blunt dissector with a larger sponge specially configured to engage and remove tissue and safely contain bleeding would have a tremendous advantage over the commonly used “peanut” dissector.

Because of the shortcomings of the “peanut” dissector, when it is necessary to stop bleeding in the surgical field, the surgeon must resort to another tool such as a laparoscopic cigarette sponge. The cigarette sponge is an absorbent sponge with a radiopaque thread that is rolled into a tight cylinder and tied at each end with umbilical tape. In the event that bleeding occurs while a surgeon is using the “peanut” dissector, the surgeon must withdraw the dissector from the patient through the trocar, introducer or cannula and then insert another tool, such as the cigarette sponge through the trocar, introducer or cannula. Such a sequence wastes valuable time. There remains a need for a blunt dissection instrument that is also effective at containing and stopping bleeding that occurs during blunt dissection.

More recently, as described in U.S. Pat. No. 5,658,307, another tool for laparoscopic surgery is a blunt dissector with an abrasive surface around the tip for manipulating and clearing tissue. The tip region has a smooth curvature to facilitate removal of the instrument from a cannula within which it may be inserted for entry into the body of the patient. The patent discloses a capture region of the tip that engages ductal and arterial structures and facilitates the dissection and removal of tissue from these structures.

However, instruments such as the dissector of the '307 patent have several disadvantages. First, the tip is not composed of expandable sponge material. Rather, the surface or coating of the tip is made of a superabrasive material such as natural or synthetic diamond, carbon films, boron nitrides, silicon carbides or ceramics. These materials may be rough or injurious to delicate tissue and may not be soft enough to avoid injury to internal organs. Because it lacks absorbent sponge material, the dissector of the '307 patent is not effective at absorbing blood. Furthermore, the tip structure of the dissector of the '307 patent is not specially configured to be maximally efficient for clearing away tissue. The curved tip will not provide enough traction and will not cause tissue to adhere to it to effectively pull the tissue away to clear the surgically important structures. Finally, the dissector of the '307 patent lacks a specially configured region for engaging ducts and vessels. The curvature of the tip's capture region does not sufficiently engage ductal and arterial structures.

Thus, although there are various surgical instruments for clearing tissue and absorbing blood in surgical procedures, there remains a need for an instrument composed of a soft and noninjurious material that can function efficiently as a blunt dissector to quickly and effectively clear tissue away from surgically important structures, engage ductal and arterial structures and also be used to contain bleeding in the surgical field.

### SUMMARY OF THE INVENTION

The invention is a surgical instrument comprising an expandable sponge. The instrument of this invention may be found to have a variety of uses during surgery, including, for example, to clear tissue away from surgically important structures and to effectively contain bleeding during surgery. The instrument of this invention provides advantages over other surgical instruments such as blunt dissectors by providing for the rapid and effective clearing of tissue and exposing surgically important structures during surgery. The surgical instrument of this invention also has the advantage of being an effective instrument for containing blood flow. The instrument has the ability to tamponade a bleeding vessel of almost any size. As a result, this invention has the ability to save valuable time during surgery. The present invention is also safer than previous surgical instruments used for similar purposes.

The invention comprises an expandable sponge which has a first unexpanded state and a second expanded state. Exposure to moisture causes the transition to the expanded state. The sponge, in its expanded state, is thick and soft and may be configured for the desired surgical procedure to be performed, for example to clear the tissue from anatomical structures. In a preferred embodiment, the sponge has a network of ridges and grooves which increase the surface area of the sponge and provides a specialized structure to catch and remove tissue. Another embodiment of the sponge has an indented neck region which extends around the circumference of the sponge. This indented neck region is configured to complement curved structures such as ducts and blood vessels and slide along these structures to remove tissue and/or expose these structures during surgery. In another embodiment, the end of the sponge has a tip region which has a smaller network of grooves and ridges and a taper angle. The expandable sponge may have taper angles at the distal and proximal ends to facilitate entry into, and removal from, a trocar or cannula. The sponge is preferrably radiopaque so that the sponge and/or instrument can be visualized.

It is an object of this invention to provide a surgical instrument configured to more effectively remove tissue from anatomical structures, including, without limitations, those that are fragile. To that end, an embodiment of the invention is an expandable sponge with a network of ridges and grooves extending around the main body of the sponge. An embodiment also has a smaller network of ridges and grooves at the distal end region of the sponge. This network of ridges and grooves provides additional surface area to effectively engage tissue during surgery, causing the tissue to attach to the sponge and become lodged in the grooves of the sponge. Thus, tissue is efficiently removed from anatomical structures as desired by the surgeon.

Another object of the invention is to provide a surgical instrument that it is safer than existing blunt dissectors. Accordingly, an embodiment of the present invention is made of soft, noninjurious material and is thicker than most blunt dissectors. The thickness of the sponge provides additional padding to protect anatomical structures from traumatic injury during surgery. A preferred embodiment comprises a network of ridges and grooves to facilitate clearing of tissue rather than an abrasive material that could harm internal tissue, vessels or organs. The sponge has a first unexpanded state and a second expanded state. The sponge expands and softens upon contact with moisture.

It is an object of this invention to provide a surgical instrument configured to fit snugly on and slideably engage curved anatomical structures such as arteries, ducts or other vessels. An embodiment of the expandable sponge of this invention has, for example, an indented neck region designed to slideably engage such curved structures. In this way, tissue can be quickly and efficiently retracted or removed from these structures during surgery.

Another object of this invention is to provide a surgical instrument useful for containing blood flow. A preferred embodiment of the expandable sponge of this invention is composed of absorbent sponge material to absorb and contain bleeding. An embodiment of the expandable sponge of this invention has a forward terminus that can be used to apply pressure to a wound or lesion, such as from an internal organ, from which blood is escaping.

It is also an object of this invention to provide a surgical instrument that can be easily used with a trocar or cannula. An embodiment of the present invention comprises an expandable sponge with a distal taper to faciliate insertion through a trocar or cannula and a proximal taper to facilitate removal from a trocar or cannula. An embodiment of the present invention comprises an expandable sponge configured to be attached to a handle. The handle is configured to fit through a trocar or cannula and allow the surgeon to easily manipulate the instrument while it is inserted through the trocar or cannula.

### BRIEF DESCRIPTION OF THE DRAWINGS

FIG. 1 shows a perspective view of a surgical instrument in accordance with the present invention;

FIG. 2 is a side view of an embodiment of the expandable sponge of the present invention showing a straight on view of one of the ridges;

FIG. 3 is a side view of an embodiment of the expandable sponge of the present invention showing a straight on view of one of the grooves;

FIG. 4 is a top view of an embodiment of the present invention;

FIG. 5 is a bottom view of an embodiment of the present invention;

FIG. 6A shows an embodiment of the unexpanded sponge of this invention divided into regions wherein the pre-expansion filet cuts can be seen;

FIG. 6B shows and embodiment of the unexpanded sponge of this invention wherein the proximal and distal portions can be seen;

FIG. 6C shows an embodiment of the sponge of the present invention unexpanded and attached to the stick;

FIG. 6D shows a perspective view of an embodiment of the surgical instrument of the present invention wherein the sponge is in an unexpanded state and the filet cuts can be seen;

FIG. 6E shows an enlarged view of an embodiment of the unexpanded sponge of the present invention demonstrating the indented neck region prior to expansion and the spacing of the filet cuts;

FIG. 7A is a schematic diagram of an embodiment of the present invention in an unexpanded state divided into regions denoted A, B, C and D;

FIG. 7B is a schematic diagram of an embodiment of the present invention in an unexpanded state divided into regions denoted A, B, C and D and showing the spacing of the filet cuts;

FIGS. 8A-8G are embodiments of the present invention in an unexpanded state divided into regions denoted A, B, C1, C2 and D;

FIG. 9 shows a further embodiment of the present invention in which the handle portion is a hollow shaft that extends through the sponge to the end of the sponge;

FIG. 10 shows a further embodiment of the present invention with multiple expanded portions;

FIG. 11 shows an embodiment of the present invention in relation to a trocar or cannula;

FIG. 12 shows an embodiment of the present invention inserted through a trocar or cannula;

FIG. 13 shows an embodiment of the present invention partially inserted through a trocar or cannula, which penetrates the surface of a patient;

FIG. 14 shows an embodiment of the present invention inserted through a trocar or cannula, which penetrates the surface of a patient;

FIG. 15 is a perspective view of an embodiment of the present invention showing the indented neck of a sponge of the present invention against an artery or duct;

FIGS. 16-18 are perspective views of embodiments of the present invention clearing tissue during surgery;

FIG. 19 is a perspective view of and embodiment of the present invention used to contain or prevent bleeding of a blood vessel during surgery.

FIG. 20 shows a bullet-shaped embodiment of the invention attached to a handle.

FIG. 21 shows an oval-shaped embodiment of the invention attached to a handle.

FIG. 22 shows an embodiment of the attachable expandable sponge of the present invention.

### DETAILED DESCRIPTION

The surgical instrument of this invention comprises an expandable radiopaque sponge for use in medical procedures. The expandable sponge described herein may be used as a blunt dissector to clear tissue away from anatomical structures during surgical procedures. It also may be configured to clear tissue from curved structures such as vessels or ducts by slideably engaging such structures. The expandable sponge is designed to be safe for contact with fragile anatomical structures and will reduce potential injury to these structures, which include organs, ducts, vessels, etc. The expandable sponge is also designed to be used with a trocar or cannula and can be easily inserted into and removed from such instruments. In certain embodiments, it may also be used as a tamponade to absorb blood and stop or contain bleeding in body cavities.

The expandable sponge of this invention is a surgical instrument designed for use in the body cavity of a patient. In a preferred embodiment, the sponge of this invention comprises an expandable material designed to expand when inserted into a body cavity. The expandable material has a first unexpanded state and a second expanded state. In a preferred embodiment, the sponge expands to between 50-100% of its original unexpanded state, but the percent expansion varies depending on the needs of the user and can be 200% or more. The sponge expands when it comes into contact with moisture, such as the fluids of a body cavity. In the event that the body cavity is too dry to cause expansion of the sponge, the surgeon may irrigate the patient to cause expansion. As known to those of skill in the art, depending on the embodiment, the expandable sponge can be made of polyvinyl acetate (PVA), a physiologically compatible sponge material that expands when exposed to moisture. Preferably, the sponge is radiopaque so that the sponge or instrument of this invention can be visualized.

In its expanded state, a preferred embodiment of the sponge is sufficiently thick and sufficiently soft so as to safely contact tissue, delicate organs or vessels and other anatomical structures without causing injury. The sponge material provides a cushion so the sponge can remove tissue without traumatizing the anatomical structures that lie beneath the tissue. In a preferred embodiment, the sponge is highly absorbent and may be used to stop or contain bleeding. As will be described herein with reference to the drawings, to contain bleeding, the terminus of the sponge is pressed against the lesion, thereby applying direct pressure to the wound.

In an embodiment of the expandable sponge of this invention, the sponge is essentially bullet shaped, comprising a taper at the distal end of the sponge. In its expanded state, this embodiment may have a smooth and even surface to facilitate the dissection or removal of tissue from organs or ductal structures. The distal taper functions to facilitate entry of the sponge through a small opening such as in an anatomical barrier, or into a trocar or cannula. An embodiment of the sponge may have a second taper at the proximal end of the sponge. The proximal taper facilitates removal from a structure such as a trocar or cannula, which is often used in laparascopic procedures to insert other surgical instruments into the body cavity.

In some embodiments, the instrument has a handle extending along a longitudinal axis and having a proximal and distal end. The expandable sponge of this invention is of sufficient size to be attached to the handle. The expandable sponge generally attaches to the distal end of the handle. The handle can be any shape or size depending upon the needs of the user. In a preferred embodiment, the handle is cylindrical so it fits snugly into a trocar. The handle allows the surgeon to easily manipulate the sponge in a body cavity. In use, the surgeon grasps the proximal end of the handle and inserts the instrument through a trocar into a body cavity. Continuing to grasp the proximal end of the handle, the surgeon manipulates the instrument so that the expandable sponge at the distal end of the handle clears tissue away from surgically important structures in the body cavity.

In an embodiment, the sponge is attached to the handle using a biocompatible adhesive or glue that would be known to those skilled in the art. Depending on the embodiment, the glue might be methylmethacrylate or the glue sold under the brand name Dermabond. Alternatively, the sponge may be mechanically attached to the handle by methods known to those skilled in the art. Other bonding methods include, but are not limited to, ultrasonic welding, snapfits, heat welding, heat sealing, adhesive bonding and the like. In one embodiment, the sponge is attached to the handle by applying adhesive or glue only to the portions of the handle underneath the indented neck portion and the distal end of the sponge. The glue is underneath the indented neck portion to ensure that the indented neck portion doesn't expand. In another embodiment, the handle has a screw at the distal end, and the sponge is screwed onto the handle.

In its dry unexpanded state, a preferred embodiment of the sponge has slots or filet cuts extending in the direction of the longitudinal axis of the sponge. The number of filet cuts varies depending on the needs of the user. To create the design of the preferred array of ridges and grooves upon expansion, four filet cuts are made and two pairs of resulting ridges on opposite sides of the sponge are glued together to create four 90-degree angles. Upon expansion, the specially configured network of grooves and ridges will be created. To create the indented neck region, there are generally no longitudinal cuts at the region of the unexpanded sponge that becomes the neck region upon expansion. Alternatively, the neck can be made with a latitudinal cut around the circumference of the sponge at the unexpanded pre-neck region. Expansion of the sponge creates a bowing effect as the sponge generally and the ridges in particular bow outward. The unexpanded sponge may be manufactured with the filet cuts and this sponge “cap” then glued onto the handle. Alternatively, the filet cuts may be made after the sponge is glued to the handle.

In its expanded state, a preferred embodiment of the body of the sponge has a large surface area comprising a network of ridges and grooves around the circumference of the sponge with the grooves in between the ridges, such network extending along the longitudinal axis of the sponge. The shape, size and number of ridges and grooves may vary depending on the needs of the user. The ridges may range from very fine to very thick. The ridges may be square or rounded. The network may comprise any number of ridges and grooves to increase the surface area of the sponge as needed. In a preferred embodiment, this network comprises four ridges and four accompanying grooves in between the ridges.

This structure is especially designed to make the instrument more effective at clearing tissue away from surgically important structures during surgery. The network of ridges and grooves increases the surface area of the sponge and provides more traction to enhance tissue contact. As the surgeon rotates the sponge along this tissue, the tissue adheres to the ridges and grooves and gets caught in the grooves of the sponge, peeling away to clear anatomical structures. This process clears tissue very quickly and efficiently and provides the surgeon with easy access to surgically important structures that require medical attention.

An embodiment of the expandable sponge in its expanded state has an indented neck region around the circumference of the sponge. This region is specially designed to clear tissue from curved structures such as blood vessels or ducts. As will be described in detail herein and with reference to the drawings, the indented neck region lies between the main body and the distal end portion of the sponge. At the neck region, the thicker portions of the sponge proximal and distal of the neck become narrow and thin to provide a cradle-like structure that fits snugly on a curved surface. The surgeon can therefore easily engage a blood vessel or duct in the cradle of the indented neck and slide the sponge along the curved surface to quickly and efficiently clear tissue from the curved surface during surgery. The sponge may have multiple indented neck regions, and the number of such regions depends on the needs of the user. A preferred embodiment has at least one indented neck region. In a preferred embodiment this indented neck region is located toward the distal end of the sponge proximal to the distal end region of the sponge.

An embodiment of the sponge also has a distal end region which is divided from the main body of the sponge by the indented neck region. The distal end region may have a network of ridges and grooves that continues in the same pattern as the network on the sponge body. In an embodiment of the invention, the ridges and grooves of the distal end region are smaller than those of the sponge body and diminish towards the distal tip of the sponge at the taper angle of the distal tip. Like the network on the sponge body, the network of ridges and grooves on the distal end region my vary in shape, size and number.

In some embodiments of the invention, the sponge has an angle where the indented neck region transitions to the distal end region of the sponge. This angle is created by the expansion of the distal end region of the sponge and comprises the indentation of the indented neck region. In a preferred embodiment, the angle of the indented neck region in relation to the distal end region is almost 90 degrees, but the neck angle will vary to suit different surgical structures. The distal tip of the sponge has a terminus that can be used to clear tissue or to apply direct pressure to cuts or tears in blood vessels to contain and stop bleeding during surgery and will be effective for small, medium and large arteries. Any part of the sponge may be used as a tamponade to contain bleeding, but head-on application of the terminus provides the most direct pressure to the lesion.

The sponge varies in size depending on the needs of the user. It can be small and very fine for use in small body cavities, or it can be larger for use in large body cavities such as the abdominal cavity. The expandable sponge has a length which extends along the longitudinal axis of the handle. The sponge also has a latitudinal axis and circumference. One embodiment of the sponge in its unexpanded state has a width of about 2 centimeters and a length of about 5 centimeters. In some embodiments, the distal end of the sponge has a 0.5 centimeter overhang past the distal tip of the handle portion of the instrument. In an embodiment of the invention, the unexpanded sponge tip is 4 centimeters wide and 10 centimeters long. In one embodiment, the unexpanded sponge has a thickness of 1.0 mm, and the wet, expanded sponge has a thickness of 16 mm or 1.6 cm.

The handle can also be various shapes such as hexagonal or octagonal depending on the needs of the user or to make the end easier for a surgeon to grasp. In a preferred embodiment, the handle is cylindrical in shape because this allows the proximal end of the handle to fit snugly through most trocars or cannulae. The proximal end of the handle may have a flattened end portion for ease of handling by the surgeon. The handle portion of the invention varies in size depending on the needs of the surgeon and the size of the patient. One embodiment of the handle has a diameter of between about 0.2 cm or 2.0 mm and about 2.0 cm or 20 mm, but can be made to fit through trocar or cannula port of any size. An embodiment of the handle has a length of between about 5 inches or 12.7 cm and about 24 inches or 60.96 cm, but can be any size depending on the size of the patient and the needs of the surgeon. The distal end of the handle portion may be shaved to reduce the diameter. Because of the reduced diameter of the shaved distal end of the handle, the distal end of the handle may be flush and contiguous with the proximal end of the sponge. Thus, in some embodiments there is no overhang of the tapered proximal end of the sponge where it meets the handle portion.

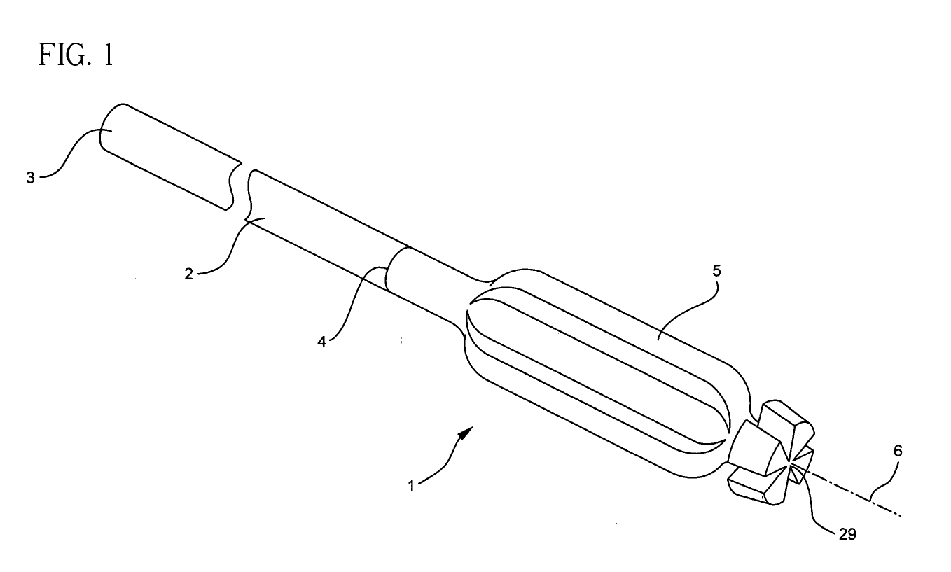

Adverting to the drawings, FIG. 1 illustrates an embodiment of the surgical instrument represented generally at 1. This instrument comprises a cylindrical handle 2 which has a proximal end 3 and a distal end 4 with an expandable sponge 5 at the distal end. The expandable sponge at the distal end extends along a longitudinal axis 6. The distal terminus of the sponge is represented here at 29. An embodiment of the sponge 5 is shown here in its expanded state.

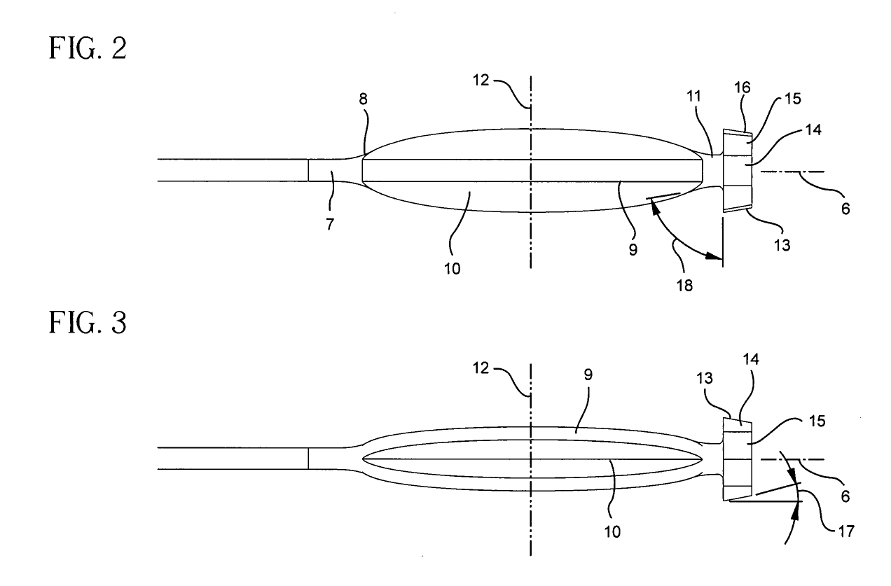

FIGS. 2 and 3 illustrate the expanded sponge in greater detail. The sponge has a proximal end region 7 with a taper angle 8 to facilitate removal of the instrument from a trocar or cannula. The body of the sponge comprises ridges 9 which extend along the longitudinal axis 6 of the sponge and grooves 10 in between the ridges. This network of ridges 9 and grooves 10 increases the surface area of the sponge so the instrument grabs and clears tissue more effectively during surgery. The increased surface area enhances tissue contact and provides more traction for clearing tissue.

The sponge has an indented neck region 11 which extends around the circumference of the sponge. This indented neck 11 functions to gently slideably engage blood vessels or ductal surfaces and safely clear tissue during surgery. The indented neck 11 is shaped to engage a curved structure such as a blood vessel. The instrument can be moved along axis 12 and rotated about axis 6 to engage and remove tissue from anatomical structures. When the indented neck 11 is placed on a duct or vessel, one sweep along the vessel will usually suffice to clear the tissue off and expose the duct or vessel.

As can also be seen in FIGS. 2 and 3, the distal end region 13 of the sponge has a small network of ridges 14 and grooves 15 and a taper 16 which functions to facilitate placement through a trocar or cannula. In a preferred embodiment, this distal end region taper angle 17 is about 5 to 30 degrees. The distal end region is divided from the main body of the sponge by the indented neck region 11. In this embodiment, the angle of the indented neck region 11 in relation to the distal end region is almost 90 degrees, but the neck angle 18 will vary to suit different surgical structures. The network of ridges 9 and grooves 10 that characterizes the body of the sponge is interrupted by the indented neck region 11, but the distal end region has a smaller network of ridges 14 and grooves 15 that continues in the same pattern as the network on the sponge body. The ridges 14 and grooves 15 of the distal end region are smaller than those of the sponge body and diminish towards the distal tip of the sponge at the taper 16 of the distal tip.

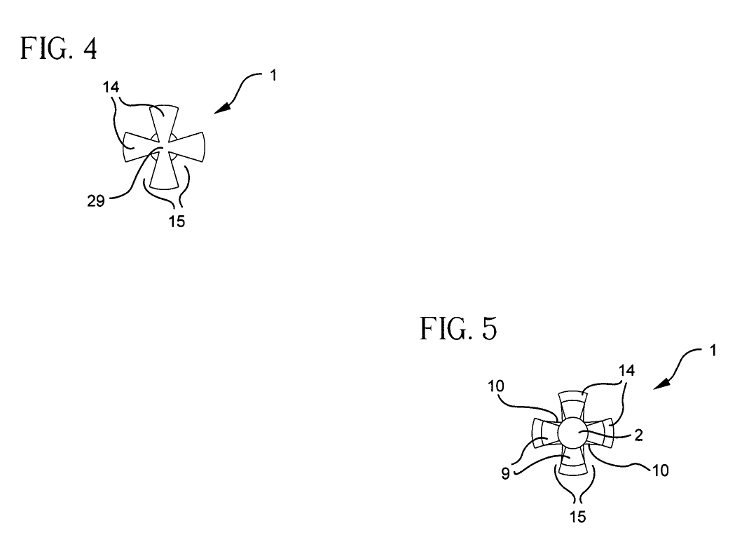

Turning to FIG. 4, this figure shows a top view of the distal end of the sponge at the forward terminus of the sponge. The small network of ridges 14 and grooves 15 can be seen in FIG. 4. The forward terminus of the sponge 29 can be seen. FIG. 5 shows a bottom view of an embodiment of the invention, wherein the circular bottom of the handle can be seen. The large ridges 9 and grooves 10 and small ridges 14 and grooves 15 of the distal end region can be seen from the bottom view. The handle can be generally cylindrical as illustrated in FIG. 5 or triangular, square, rectangular, hexagonal, octagonal or other shapes as needed by the user. A cylindrical handle allows the proximal end of the handle to fit through the trocar. The proximal end of the handle may have a flattened end for ease of handling by the surgeon. The handle may vary in length, width or shape depending on the shape and dimension of the sponge.

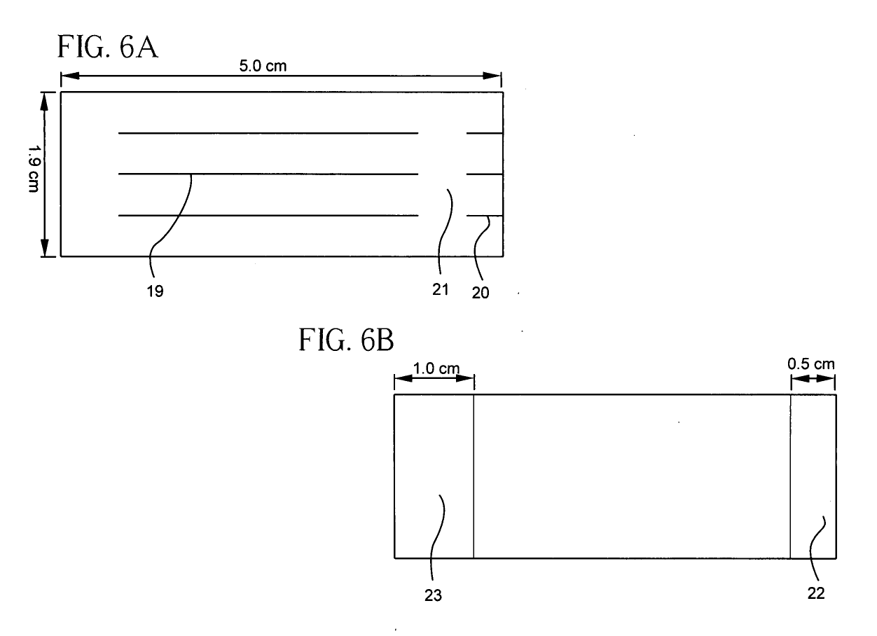

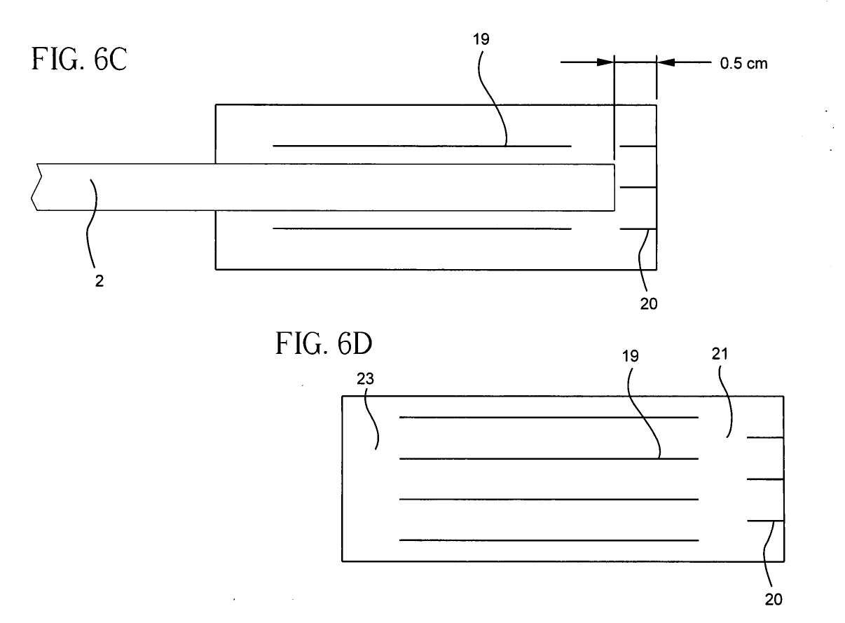

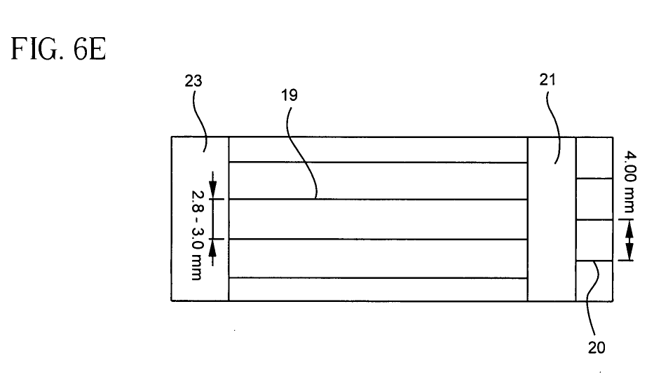

FIGS. 6A and 6B show embodiments of the invention wherein the sponge is in a dry unexpanded state. The unexpanded sponge has a smaller length and width and a smaller circumference than the expanded sponge. To create the array of ridges and grooves upon expansion, three filet cuts 19 are made to form slots on each side of the sponge and two pairs of resulting ridges on opposite sides of the sponge are glued together to create four 90-degree angles. These filet cuts 19 can be seen in FIG. 6A. The unexpanded sponge also has smaller filet cuts 20 at the distal end that form the smaller network of ridges and grooves at the distal end of the expanded sponge. As can be seen in FIG. 6A, there are no slots at the pre-neck region 21 that will expand to create the indented neck region.

FIG. 6A shows an embodiment of the unexpanded sponge of the invention wherein the width of the sponge is about 1.9 cm and the length is about 5.0 cm. In another embodiment of the invention, the unexpanded sponge is 4 centimeters wide and 10 centimeters long, which becomes 6 centimeters by 15 centimeters upon expansion.

FIG. 6B shows an embodiment of the unexpanded sponge of this invention wherein the proximal and distal portions of the sponge can be seen. In this embodiment, there is a 0.5 cm distal portion of the sponge 22 that will hang over the end of the handle portion of the instrument. Also, the 1 cm proximal portion 23 can be seen. As appreciated by those skilled in the art, the distal and proximal portions of the sponge may be shaved or sanded to create a taper angle.

The unexpanded sponge may be manufactured with the filet cuts, and this sponge “cap” then fit onto the handle. Alternatively, the filet cuts may be made after the sponge is glued to the handle. Upon expansion, these filet cuts take the form of the network of ridges 9 and grooves 10 seen in FIGS. 2 and 3. Expansion of the sponge creates a bowing effect as the sponge generally and the ridges particularly bow outward.

Turning to FIG. 6C, one can see an embodiment of the present invention wherein the sponge is unexpanded and attached to the end of the handle portion 2. One can again see the filet cuts 19 and 20 or filet effect in this figure. As can be seen in FIG. 6C, in this embodiment the distal end of the sponge has an overhang past the end of the handle portion which extends through most of the sponge. In this embodiment, an overhang past the end of the handle measures about 0.5 cm.

FIG. 6D shows an enlarged view of an embodiment of the unexpanded sponge of the present invention. Both the body slots or filet cuts 19 and the distal end region filet cuts 20 can be seen here together with the region of the sponge that is shaved 23 to form the proximal taper angle. One can also see that there are no filet cuts at the pre-neck region 21 that will become the indented neck portion when the sponge expands.

FIG. 6E shows an enlarged view of an embodiment of the unexpanded sponge of the present invention demonstrating the indented neck region prior to expansion. As can be seen more clearly in FIG. 6E, the pre-neck region 21 that will become the indented neck region upon expansion has no slots or filet cuts in unexpanded form. FIG. 6E illustrates an embodiment of the invention wherein the distance between the sponge body filet cuts 19 is about 2.8-3.0 mm. In this embodiment, the distance between the distal end region filet cuts 20 is about 4 mm. One can also see the proximal end 23 of the sponge that will become the proximal taper angle.

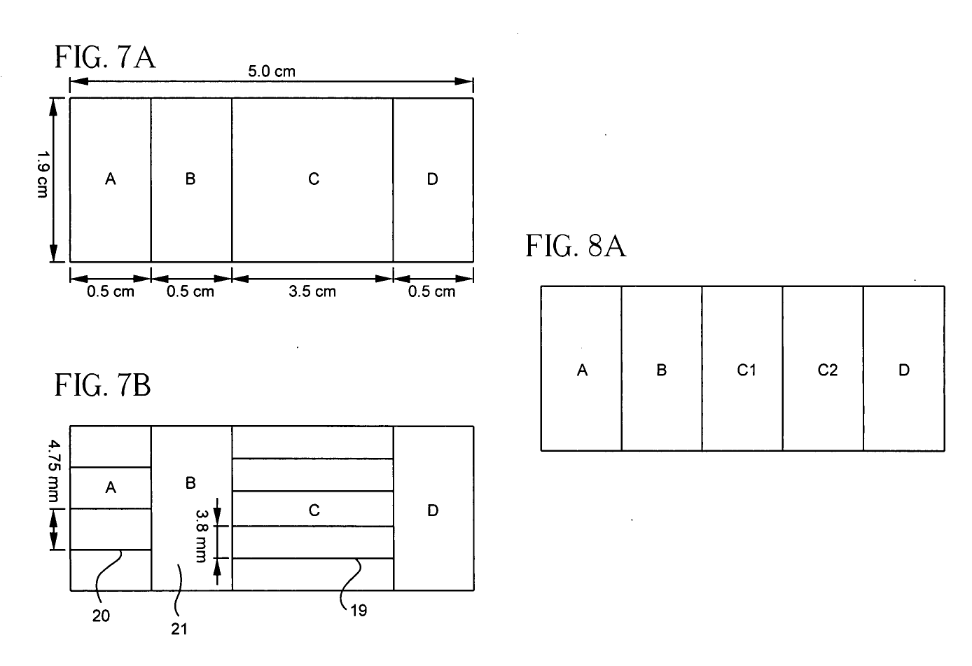

FIG. 7A shows a schematic diagram of an embodiment of the unexpanded sponge divided into regions denoted A, B, C and D. FIG. 7B shows a more detailed schematic of the unexpanded sponge also divided into regions denoted A, B, C and D wherein region A is the distal end region, region B is the pre-indented neck region, region C is the main body of the sponge and region D is the proximal region of the sponge that will form a taper angle. Region A measures 0.5 cm from the distal end to the start of region B, region B measures 0.5 cm to region C and region C is 3.5 cm to region D, which measures 0.5 cm to the proximal end of the sponge. The sponge body filet cuts 19 and the distal end region filet cuts 20 can be seen. The width of this embodiment of the sponge is about 1.9 cm or 19 mm, and the length of the sponge is about 5.0 cm or 50 mm.

As can be seen in FIG. 7B, 3 filet cuts are made in region A to create four equally sized areas. The cuts end proximal to region B, which will become the indented neck region. In region C, four filet cuts are made to create five equally sized areas. Region C requires four filet cuts to create two pairs of resulting ridges on opposite sides of the sponge that may be glued together to create the network of four grooves and ridges. A sponge with three cuts would also suffice. In this embodiment, the distance between the body filet cuts 19 is about 3.8 mm, and the distance between the distal end region filet cuts 20 is about 4.75 mm. As can be seen again in FIG. 7B, there are no filet cuts at the pre-neck region 21 that will become the indented neck portion when the sponge expands. The number of filet cuts made varies depending on the needs of the user.

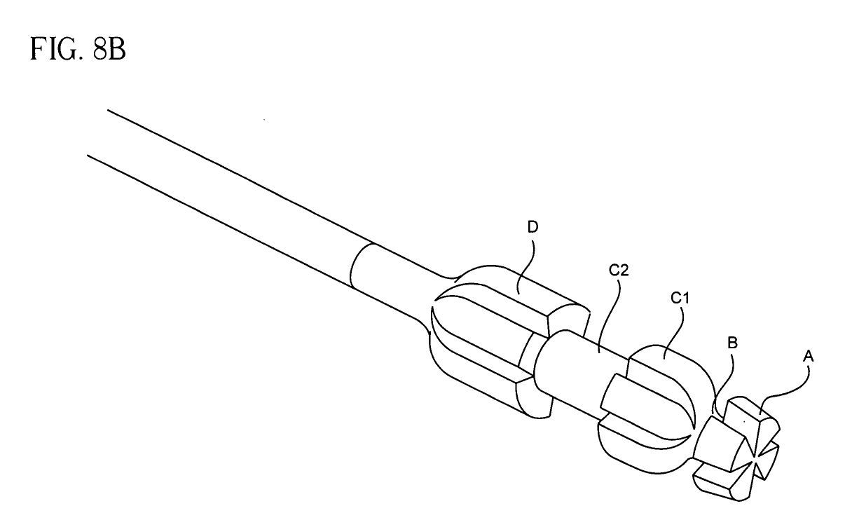

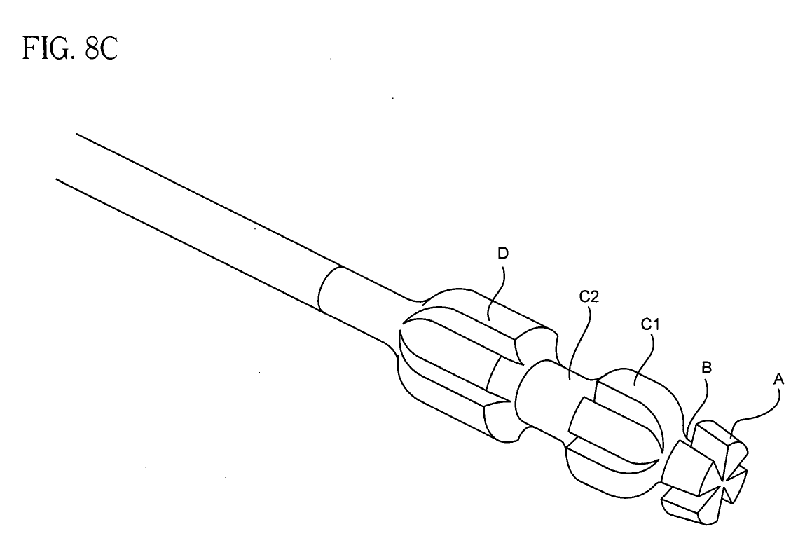

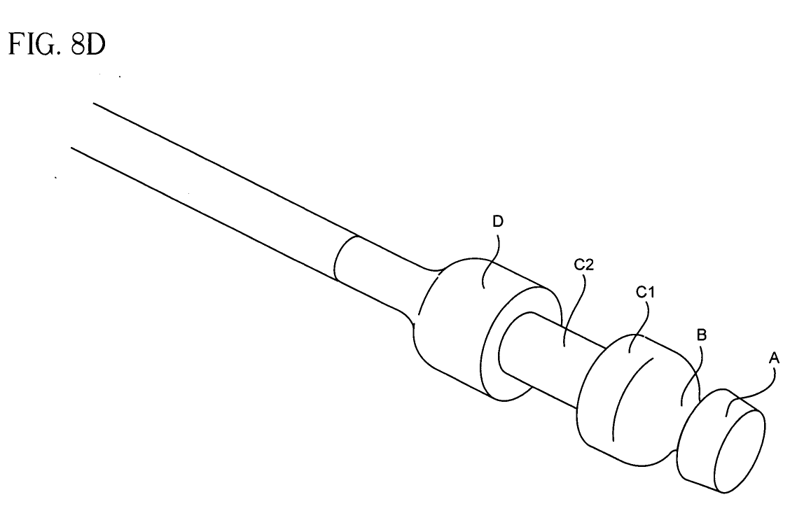

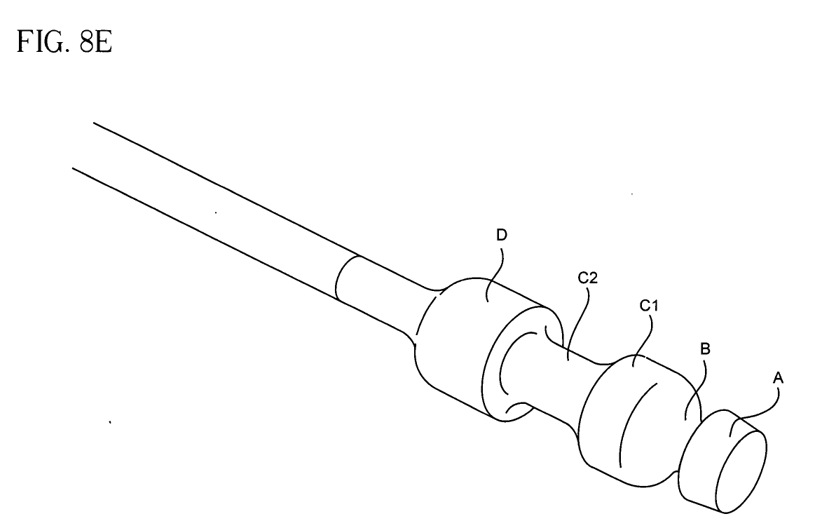

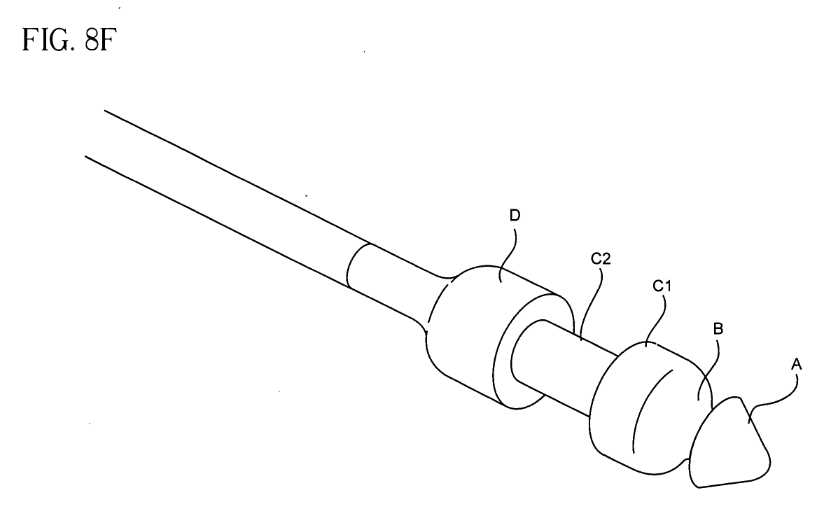

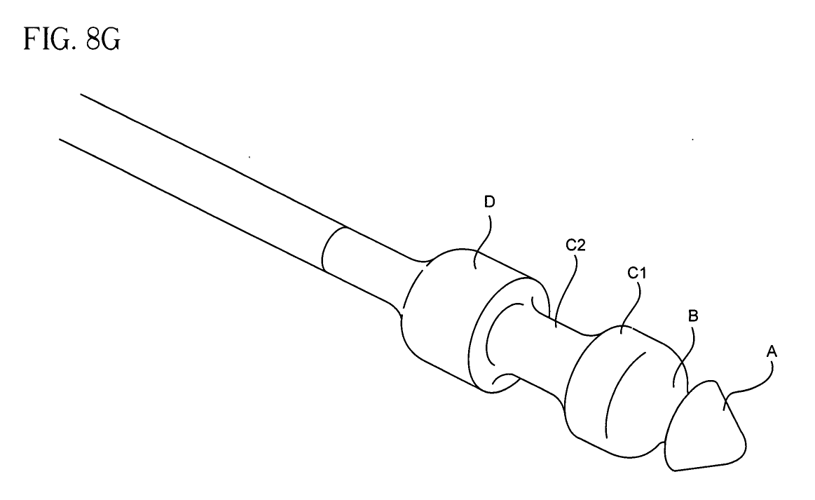

FIG. 8A shows a schematic diagram of an embodiment of the unexpanded sponge divided into regions denoted A, B, C1, C2 and D. Subsequent FIGS. 8B-8G show various embodiments of the expanded sponge with the regions still labeled as in FIG. 8A. In these figures, one can see the relationship between the regions in the unexpanded sponge and the same regions in various embodiments of the expanded sponge. In this series of figures, B=1 cm, C1=1 cm and C2=1 cm. In all of these embodiments, regions B and C2 have been cut to about half of the sponge diameter.

FIG. 8B shows an embodiment of the expanded sponge wherein regions B and C2 are squared indented neck regions cut to about one half of the sponge diameter, and regions A, C1 and D are expanded regions. In particular, region A is the distal tip region with a small network of ridges and grooves, and region D is the proximal taper angle. FIG. 8C shows another embodiment of the expanded sponge wherein regions B and C2 are tapered indented neck regions cut to about one half of the sponge diameter, and regions A, C1 and D are expanded regions. In particular, region A is the distal tip region with a small network of ridges and grooves, and region D is the proximal taper angle.

FIG. 8D shows another embodiment of the expanded sponge wherein regions B and C2 are indented neck regions cut to about one half of the sponge diameter, and regions A, C1 and D are expanded regions. In particular, region A is the distal tip region, and region D is the proximal taper angle. As can be seen in FIG. 8D, this embodiment of the sponge has no ridges or grooves. FIG. 8E shows another embodiment of the expanded sponge wherein regions B and C2 are tapered indented neck regions cut to about one half of the sponge diameter, and regions A, C1 and D are expanded regions. In particular, region A is a square-shaped distal tip region, and region D is the proximal taper angle.

FIG. 8F shows another embodiment of the expanded sponge wherein regions B and C2 are squared indented neck regions cut to about one half of the sponge diameter, and regions A, C1 and D are expanded regions. Region A forms the distal taper angle, and region D forms the proximal taper angle. Looking next to FIG. 8G, one can see another embodiment of the expanded sponge wherein regions B and C2 are tapered indented neck regions cut to about one half of the sponge diameter, and regions A, C1 and D are expanded regions. Region A forms the distal taper angle, and region D forms the proximal taper angle.

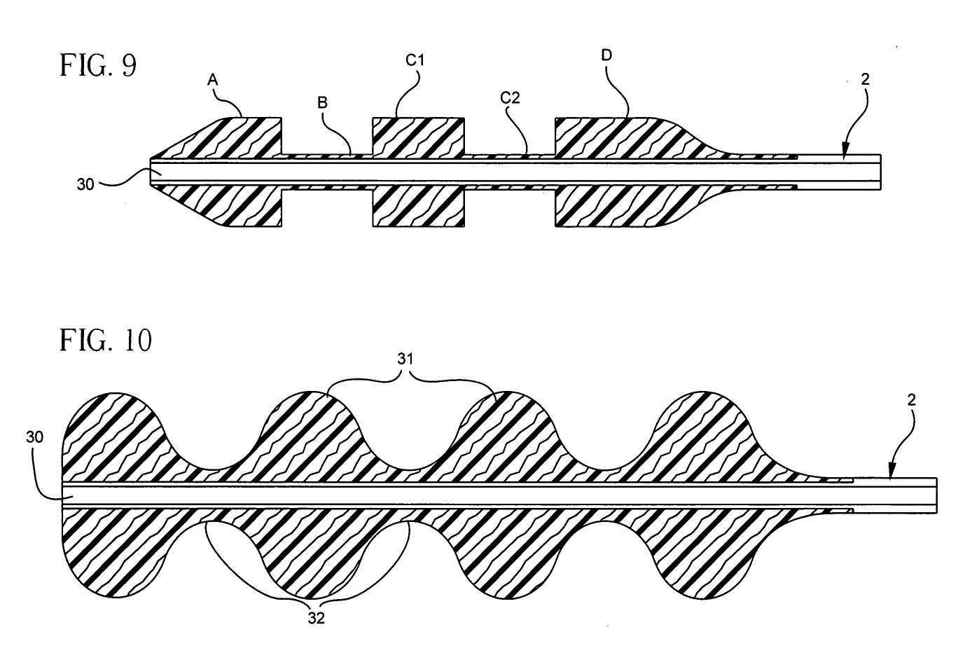

FIG. 9 shows an embodiment of the expanded sponge wherein the handle portion 2 has a hollow interior 30 that extends through the sponge to the end of the sponge. The hollow shaft may provide a ball-point pen-like apparatus with a hard tip to add a hard head, retractable instrument. In this figure, one can see the expanded sponge wherein regions B and C2 are tapered indented neck regions cut to about one half of the sponge diameter, and regions A, C1 and D are expanded regions. Region A forms the distal taper angle, and region D forms the proximal taper angle.

FIG. 10 show an embodiment of the invention with multiple expanded portions 31 and multiple tapered regions 32. The number of expanded portions and tapered regions varys depending on the needs of the user. In this figure, one can see that the handle portion 2 has a hollow interior 30.

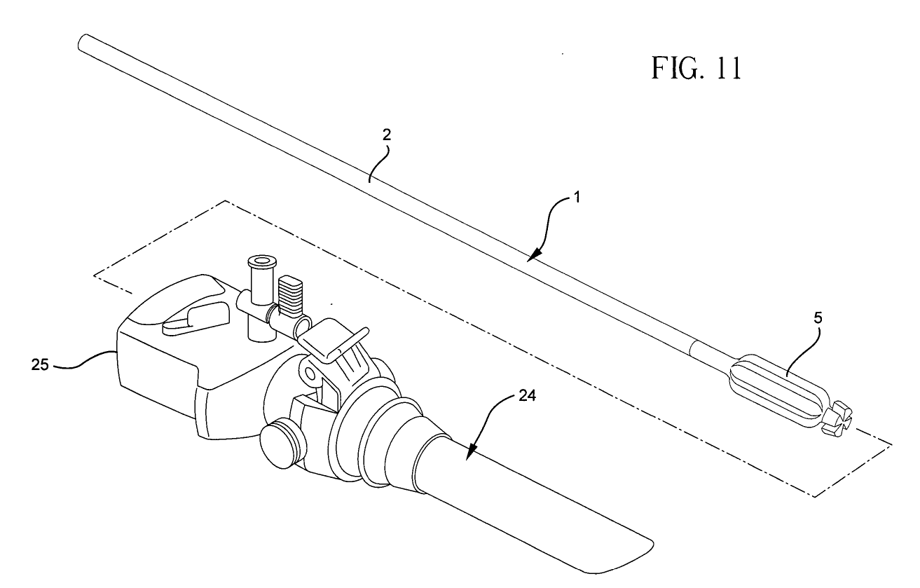

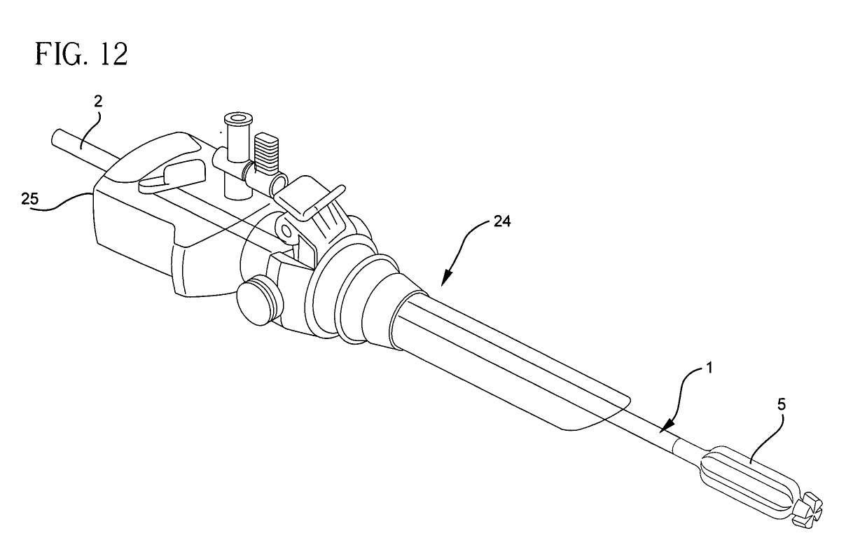

FIG. 11 shows an embodiment of the invention in relation to a trocar or cannula 24. FIG. 12 shows insertion of an embodiment of the invention through a trocar or cannula 24. The instrument with a 2×5 centimeter sponge fits through an 11-12 mm cannula port 25. The instrument can be scaled to fit through ports of varying sizes, including a 10 mm cannula port. The distal handle end of the instrument extends outside the cannula port 25 so the surgeon may manipulate the instrument. The expandable sponge 5 extends into the surgical cavity of the patient.

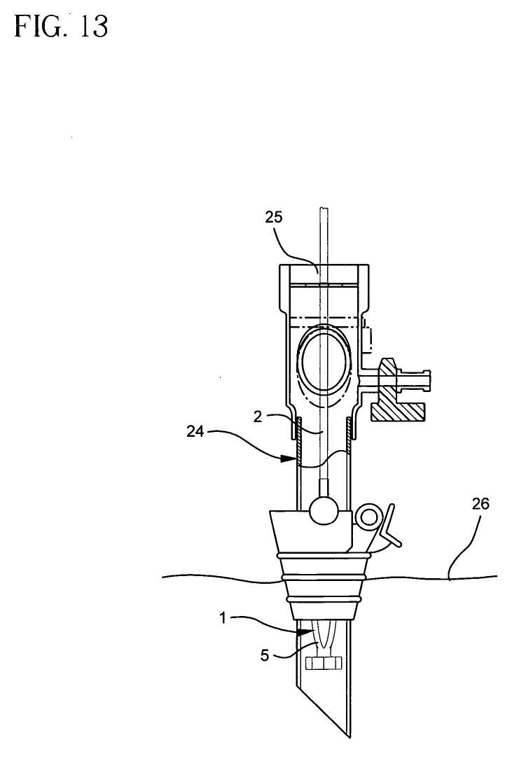

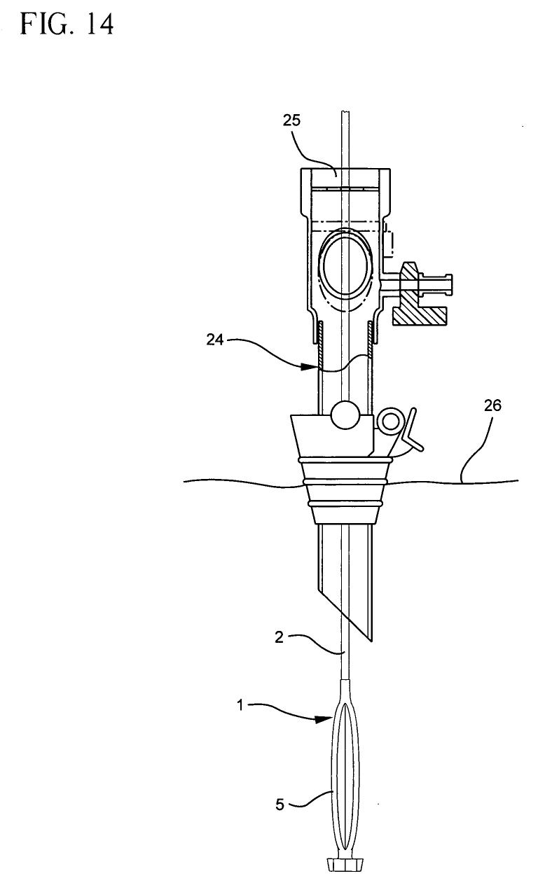

FIGS. 13 and 14 show insertion of an embodiment of the invention through a cannula and through the skin 26 of a patient. As can be seen in FIGS. 13 and 14, the distal handle end of the instrument extends outside the cannula port 25 so the surgeon may manipulate the instrument. The expandable sponge 5 extends into the surgical cavity of the patient.

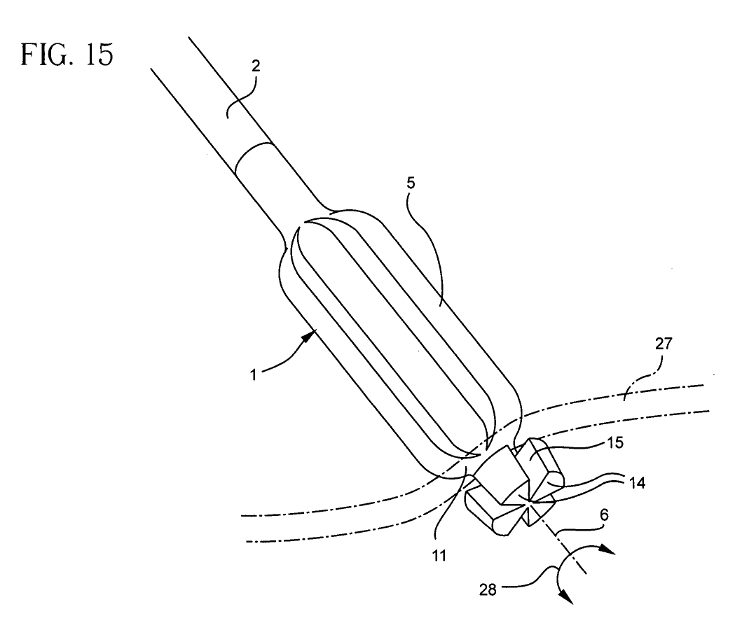

FIG. 15 shows a representative method of the invention to clear tissue from curved structures such as blood vessels or ducts during surgery. As can be seen in FIG. 15, the indented neck region 11 is shaped such that the surgeon may rest this region of the sponge comfortably on a blood vessel or duct, represented here at 27, and slideably engage this structure. The surgeon then slides the instrument along the vessel or duct while spinning the instrument about axis 6 and around arc 28 360 degrees in either direction. This combination of sliding and spinning along the curved structure with the structure disposed in the indented neck region safely and efficiently clears tissue away from such structures.

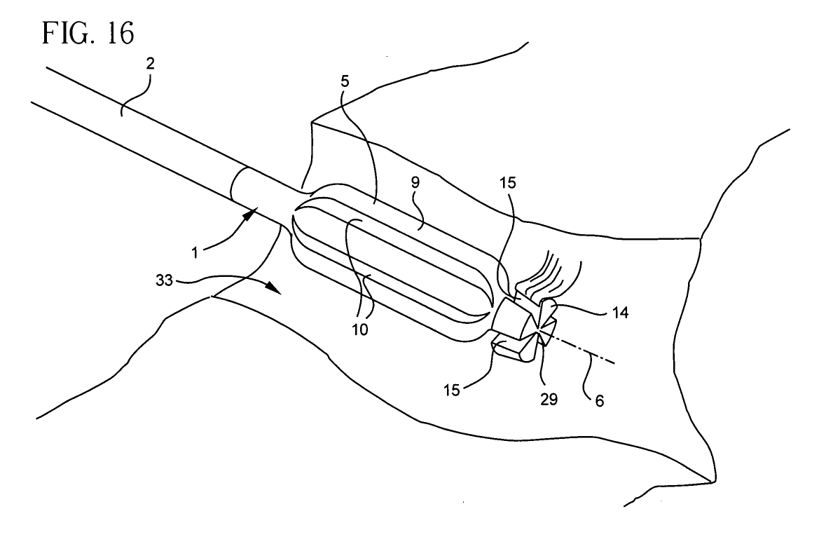

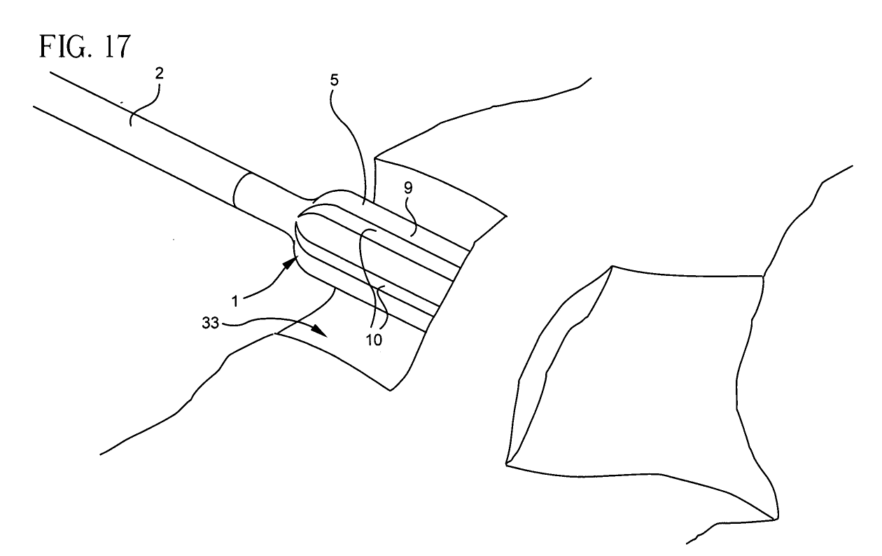

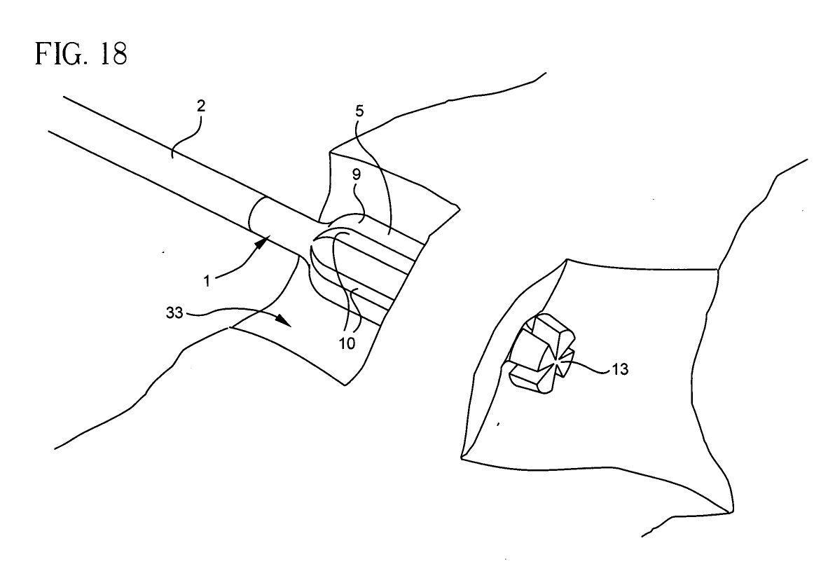

FIGS. 16-18 show use of an embodiment of the present invention manipulating tissue 33 in surgery. Investment tissue may comprise aerolar, mesentery, adhesive or connective tissue. It may also include peritoneum membranous tissue or adipose tissue. As can be seen from FIG. 16, the instrument is rotated about axis 6, and the spinning causes fatty tissue and other tissue to get caught in the grooves 10 of the sponge. This rotation and sliding of the instrument is very effective at removing the tissue and exposing surgically important structures as the network of ridges 9 and grooves 10 contacts the tissue. Fatty tissue can also be pushed away using the small ridges 14 and grooves 15 of the distal tip of the sponge. FIGS. 17 and 18 show further use of an embodiment of this invention to clear tissue during surgery. In FIG. 17 an embodiment of the invention 1 can be seen inserted into tissue. In FIG. 18, one can see the distal end region 13 of the sponge 5 inserted into tissue 33.

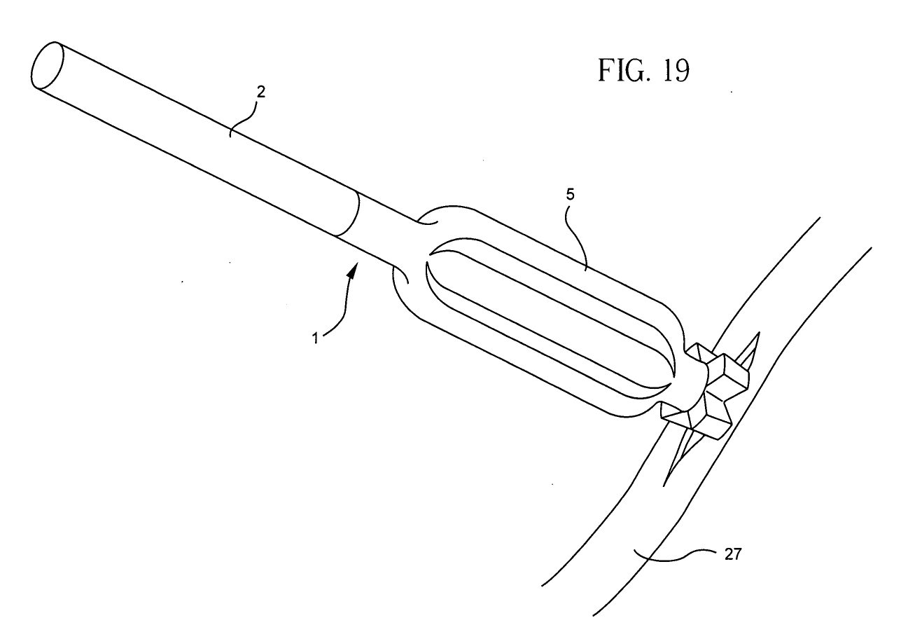

FIG. 19 shows use of an embodiment of the present invention during surgery to contain or stop the bleeding of a blood vessel 27. In the event of blood vessel bleeding where the vessel can't immediately be clipped or tied, a surgeon can use the present invention as a tamponade to apply direct pressure to the site of the bleeding while preparing to clip or tie the site. The terminus 29 at the distal tip of the sponge, which can best be seen in FIG. 1, is firmly pressed against the source of the bleeding. Any part of the sponge may be used as a tamponade to contain bleeding, but head-on application of the terminus provides the most direct pressure to the lesion. The present invention can be used to contain bleeding in any surgery where visualization of the surgical field may be blocked by leaking blood vessels. A larger embodiment of the present invention would be useful to stop a leaking abdominal aortic aneurysm or to treat a stab wound to the heart. This invention could even be used by doctors to begin treatment of these conditions in the emergency room. A doctor could make an incision on the patient in the emergency room and use the invention to stop the bleeding.

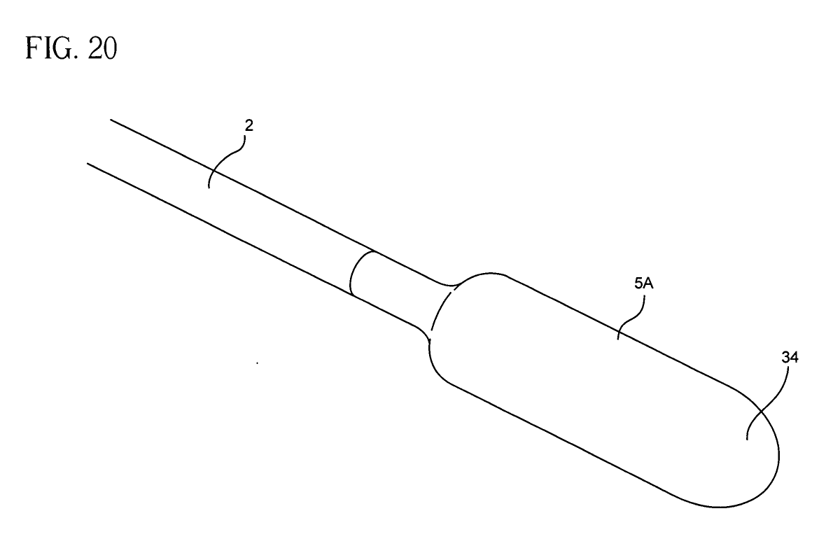

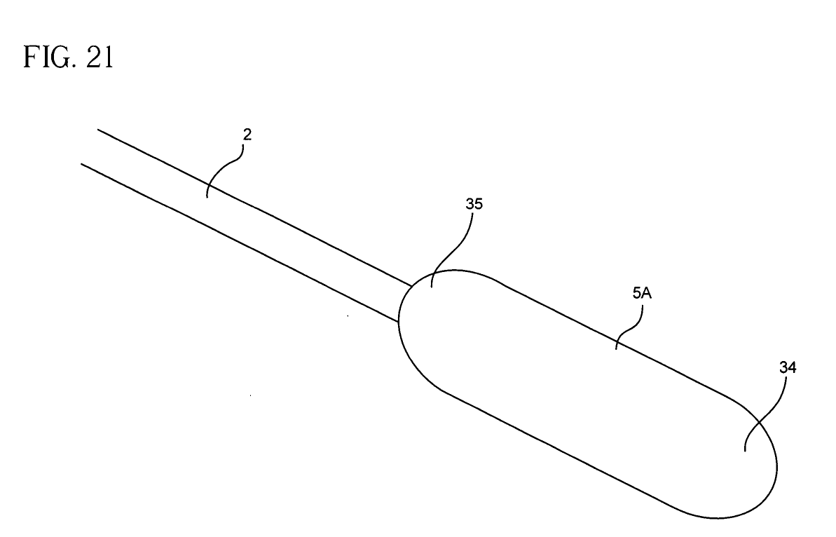

FIG. 20 shows an embodiment of the invention wherein the expandable sponge 5A has a taper 34 at the distal portion and is attached to handle 2. This is essentially a bullet-shaped sponge. In this embodiment the expandable sponge has a smooth and even surface and lacks the complex ridge and groove structure that characterizes other embodiments of this invention. FIG. 21 shows an embodiment of the invention wherein the expandable sponge 5A has a taper 34 at the distal portion of the sponge and a taper 35 at the proximal portion of the sponge.

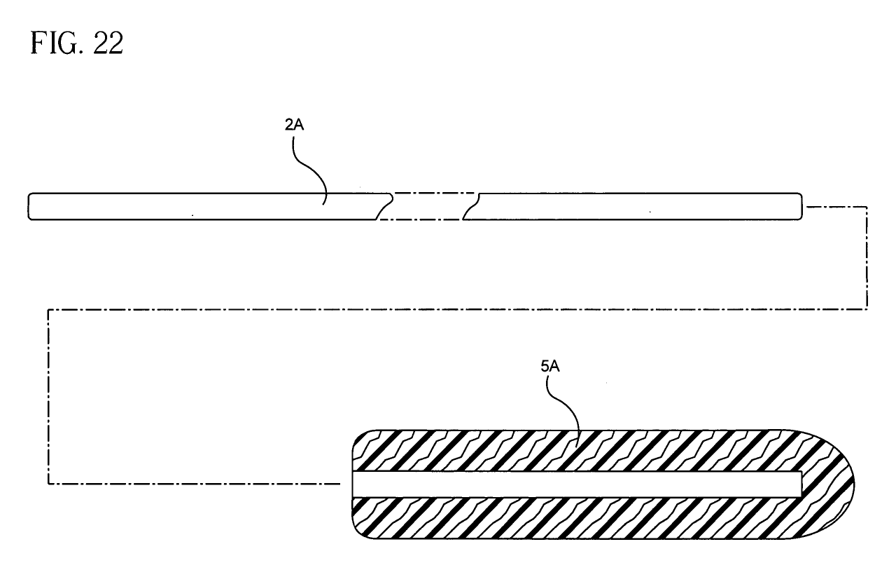

FIG. 22 shows an embodiment of the expandable sponge 5A of the present invention. This is a bullet-shaped sponge with a smooth and even surface. One can also see a handle 2A to which the sponge may be attached. In this figure, one can see the hole in the sponge where the handle is inserted to attach the sponge to the handle. In some embodiments, the material comprising the inner surface of this hole provides additional firmness to aid in attachment.

The present invention can be used for blunt dissection in all laparoscopic surgical procedures including, but not limited to, thoracic procedures and cholycystectomies. The present invention is useful in gynecological surgery and general abdominal surgery or any surgery where the surgeon needs to clear tissue away from blood vessels and other organs. The invention can also be used to prepare vessels for clipping.

It should be understood that the above description only represents illustrative examples of embodiments. For the reader's convenience, the above description has focused on a representative sample of all possible embodiments, a sample that teaches the principles of the invention. Other embodiments may result from a different combination of portions of different embodiments. The description has not attempted to exhaustively enumerate all possible variations.

Furthermore, since numerous modifications and variations will readily occur to those skilled in the art, it is not desired that the present invention be limited to the exact construction and operation illustrated. Accordingly, all suitable modifications and equivalents that may be resorted to are intended to fall within the scope of the claims.

What is claimed is:

1. A surgical instrument comprising:
an expandable sponge suitable for insertion into a body cavity;
the expandable sponge having a proximal and distal portion and a taper at the distal portion; and
a handle having a proximal and distal end wherein the expandable sponge is attached to the distal end;
wherein the expandable sponge comprises a material having a first unexpanded state and a second expanded state.

2. The instrument of claim 1 wherein the expandable sponge further has a taper at the proximal portion of the sponge.

3. The instrument of claim 1 wherein the expandable sponge further has at least one indented neck region having a shape suitable for engaging curved surfaces.

4. The instrument of claim 1 wherein the expandable sponge in the first unexpanded state further has slots extending about the sponge in a longitudinal direction or radial direction.

5. The instrument of claim 4 wherein in the second expanded state the slots expand to form a network of ridges and grooves on the expandable sponge.

6. The instrument of claim 1 wherein the taper at the distal portion of the sponge has an angle of between about 5 to 30 degrees.

7. The instrument of claim 5 wherein the network of ridges and grooves has four ridges and four grooves around the circumference of the sponge.

8. The instrument of claim 3 wherein the indented neck region extends around the circumference of the sponge.

9. The instrument of claim 3 wherein the distal portion of the sponge and the indented neck region form an angle in relation to each other of up to about 90 degrees.

10. The instrument of claim 1 wherein the sponge is generally dehydrated in the unexpanded state.

11. The instrument of claim 1 wherein the sponge is expanded by exposure to moisture.

12. The instrument of claim 1 wherein the expandable sponge expands to at least about 150%-200% of its unexpanded size in at least one dimension.

13. The instrument of claim 1 wherein the handle has a cross section that is round, square, rectangular, triangular, hexagonal, octagonal, polygonal or any combination thereof.

14. The instrument of claim 1 wherein the handle is hollow.

15. The instrument of claim 1 wherein the unexpanded sponge has a length of about 5 centimeters and a width of about 2 centimeters.

16. The instrument of claim 1 wherein the unexpanded sponge has a length of about 10 centimeters and a width of about 4 centimeters.

17. The instrument of claim 1 wherein the expanded sponge has a length of about 15 centimeters and a width of about 6 centimeters.

18. The instrument of claim 1 wherein the distal end of the handle further has a flattened portion.

19. The instrument of claim 1 wherein the instrument is a sterile single use instrument.

20. A surgical instrument comprising:
an expandable sponge suitable for insertion into a body cavity;
the expandable sponge having a proximal and distal portion and a taper at the distal portion;
wherein the expandable sponge comprises a material having a first unexpanded state and a second expanded state; and
wherein the expandable sponge has an attachment region configured for attachment to a handle.

21. The surgical instrument of claim 20 wherein the attachment region is configured to screw onto a handle.

22. The surgical instrument of claim 20 wherein the attachment region is configured to snap fit onto a handle.

23. A surgical instrument comprising:
an expandable sponge suitable for insertion into a body cavity;
the expandable sponge having a proximal and distal portion and a taper at the distal portion;
a handle having a proximal and distal end wherein the expandable sponge is attached to the distal end;
wherein the expandable sponge comprises a material having a first unexpanded state and a second expanded state;
the expandable sponge further having at least one indented neck region having a shape suitable for engaging curved surfaces;
the expandable sponge in the first unexpanded state having slots extending about the sponge in a longitudinal direction or radial direction; and
the expandable sponge in the second expanded state having the slots expanded to form a network of ridges and grooves.

24. A method of using the surgical instrument of claim 23 to clear tissue during surgery comprising:
inserting the surgical instrument into an individual;
manipulating the surgical instrument so that the expandable sponge at the distal end of the handle comes into contact with tissue;
clearing the tissue by rotating the sponge along its longitudinal axis and moving the sponge along its latitudinal axis.

25. The method of claim 24 further comprising engaging curved structures in the indented neck region and sliding the indented neck region along the curved structures to clear away any tissue associated with the curved structures.

26. A method of using the surgical instrument of claim 24 to contain bleeding during surgery comprising:
inserting the surgical instrument into an individual;
manipulating the surgical instrument so that the distal portion of the sponge comes into contact with a source of bleeding.

27. An expandable sponge suitable for insertion into a body cavity comprising:
a proximal and distal portion and a taper at the distal portion;
a material having a first unexpanded state and a second expanded state.

28. The expandable sponge of claim 27 having a generally bullet-like shape.

29. The expandable sponge of claim 27 further having a taper at the proximal portion and having a generally oval-like shape.

30. The expandable sponge of claim 27 further including ribs.

31. The expandable sponge of claim 27 wherein the ribs are along a longitudinal axis of the sponge.

32. The expandable sponge of claim 27 further including an indented neck region for engaging curved surfaces.

33. The expandable sponge of claim 27 further including an arrow-like portion on the distal end portion of the sponge.

34. The expandable sponge of claim 27 wherein the sponge is made of a physiologically compatible sponge material.

35. The expandable sponge of claim 27 wherein the material expands when exposed to moisture.

36. The expandable sponge of claim 27 wherein the sponge has a clip mechanism for attachment to a handle.

37. The expandable sponge of claim 27 wherein the sponge has an attachment region configured to screw onto a handle.
